(19) United States
(12) Patent Application Publication
(10) Pub. No.: US 2009/0310737 A1
(43) Pub. Date: Dec. 17, 2009

(54) METHOD FOR COMPUTER TOMOGRAPHY, AND COMPUTER TOMOGRAPH

(75) Inventors: Peter Forthmann, Hamburg (DE); Thomas Koehler, Norderstedt (DE); Robert Manzke, Cambridge, MA (US); Michael Grass, Buchholz in der Nordheide (DE); Andy Ziegler, Hamburg (DE)

Correspondence Address: PHILIPS INTELLECTUAL PROPERTY & STANDARDS, P. O. Box 3001, BRIARCLIFF MANOR, NY, 10510, US

(73) Assignee: KONINKLIJKE PHILIPS ELECTRONICS N.V., Eindhoven (NL)

(21) Appl. No.: 11/721,543

(22) PCT Filed: Dec. 13, 2005
(86) PCT No.: PCT/IB05/54198

§ 371 Date: Jun. 13, 2007

(30) Foreign Application Priority Data
Dec. 22, 2004 (EP) 04106890.9

(51) Int. Cl. A61B 6/00

(52) U.S. Cl. 378/8; 378/13; 378/11

(57) ABSTRACT

According to the invention, there is provided a method of recording images of the heart in computer tomography, in which, in order to prevent movement artifacts, the images are reconstructed on the basis of similar movement states of the heart and different radiation intensities are used for different movement states. Also provided is a computer tomograph for recording images of the heart in computer tomography by means of time windows which exhibit similar movement states of the heart in order to prevent movement artifacts, said computer tomograph comprising a control device which controls a radiation source with different radiation intensities for different movement states. Furthermore provided is a computer program for a computer tomograph for recording images of the heart in computer tomography by means of time windows which exhibit similar movement states of the heart in order to prevent movement artifacts, and for controlling a radiation source with different radiation intensities for different movement states.

The invention relates to a method for computer tomography as claimed in the preamble of claim 1, to a computer tomograph as claimed in the preamble of claim 13 and to a computer program as claimed in the preamble of claim 14.

In the field of computer tomography, use is made inter alia of spiral methods in which a radiation source and a detector device are moved around an object in a spiral or helical path, and the radiation transmitted through the object is detected by a detector device. This will be referred to below as spiral computer tomography. The object is in this case usually a patient to be examined or part of said patient. The spiral path is achieved by moving the radiation source in a circular manner around the object while simultaneously moving the object within the circular path, perpendicular to the plane defined by the circular path. Especially when recording moving organs, such as the heart for example, in order to prevent movement artifacts use is sometimes made only of data recorded along the spiral path of the radiation source and the detector device which exhibit the same movement state of the organ. Movement artifacts are image errors due to recordings of different movement states of the object, in this case a moving organ such as the heart. When reconstructing images from the recorded data of a detector device, in this case use is therefore made only of incomplete recorded data of the same movement states, whereas other data which are recorded in different movement states are screened out or not used. The recorded data from the detector path are therefore not used to create or reconstruct the image for all locations of the detector device along its circular or spiral path, but rather only recorded data from individual segments of the detector path are used and recorded data which lie outside these segments do not contribute to the imaging. This method comprising the use of recorded data from identical movement states of the object is referred to as gating.

It is an object of the invention to provide an improved gating method.

According to the invention, this object is achieved by the features of claims 1, 13 and 14.

According to the invention, there is provided a method of recording images of the heart in computer tomography, in which, in order to prevent movement artifacts, the images are reconstructed on the basis of similar movement states of the heart and different radiation intensities are used for different movement states. Also provided is a computer tomograph for recording images of the heart in computer tomography by means of time windows which exhibit similar movement states of the heart in order to prevent movement artifacts, said computer tomograph comprising a control device for controlling a radiation source with different radiation intensities for different movement states. Furthermore provided is a computer program for a computer tomograph for recording images of the heart in computer tomography by means of time windows which exhibit similar movement states of the heart in order to prevent movement artifacts, and for controlling a radiation source with different radiation intensities for different movement states. The radiation dose to which the patient and the operating staff of the computer tomograph are exposed is significantly reduced as a result.

Embodiments of the invention are described in the dependent claims.

The invention is particularly suitable for a prospective gating method in which different radiation intensities are used without knowing the actual movement states of the heart.

The invention will be further described with reference to examples of embodiments shown in the drawings to which, however, the invention is not restricted.

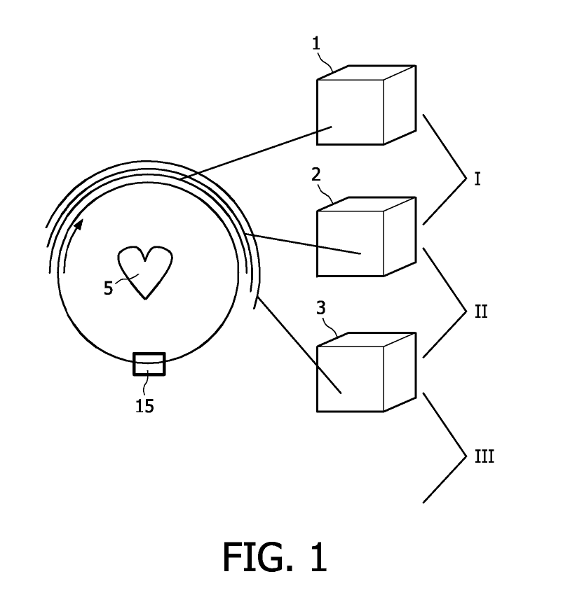

FIG. 1 schematically shows a rotation of a radiation source of a computer tomograph in a circular path around a heart with three different time windows, and also the heart volumes which are reconstructed in these time windows.

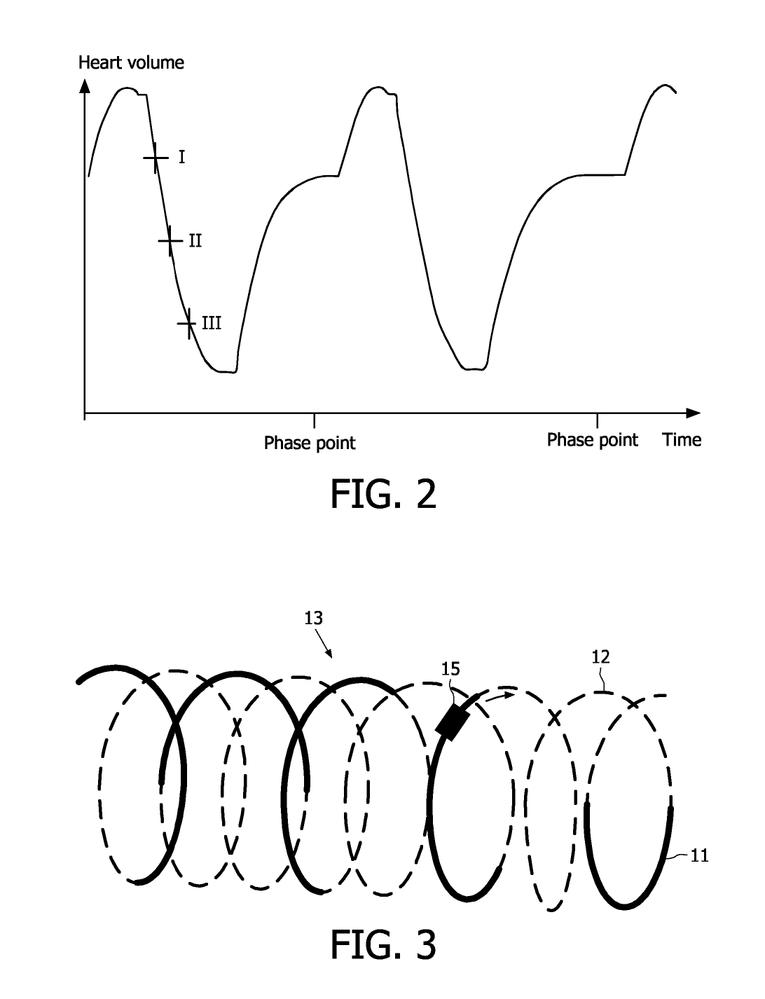

FIG. 2 shows allocations of the time windows of FIG. 1 to a curve which represents beating movements of the heart, in terms of the heart volume as a function of time, wherein the movement between the time windows can be seen.

FIG. 3 shows a spiral path of a radiation source of the computer tomograph, in which the time windows are identified by bold spiral path segments.

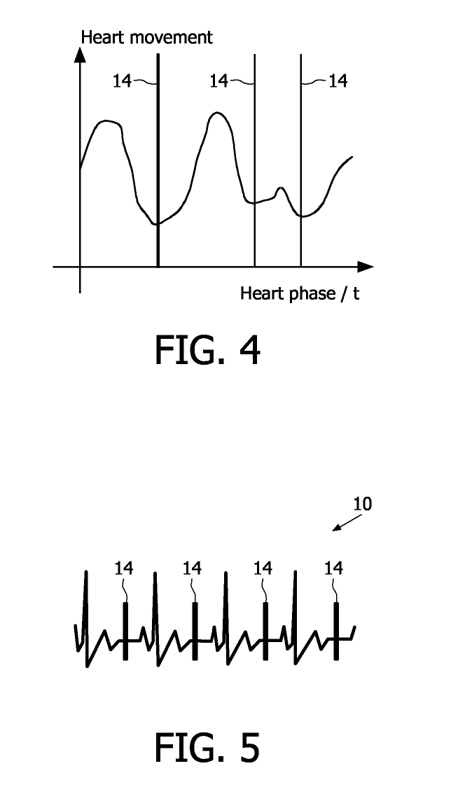

FIG. 4 shows a diagram of a heart movement as a function of a heart phase.

FIG. 5 shows an electrocardiogram with associated heart phases as shown in FIG. 4.

FIG. 1 schematically shows a rotation of a radiation source 15 of a computer tomograph about a circular path in the direction of the curved arrow. The radiation source 15 usually moves around an examination object, in this case a heart 5, and transmits X-ray radiation essentially in the direction of the heart 5, said X-ray radiation being picked up by a detector device of the computer tomograph that is located opposite the radiation source 15. The radiation source 15 may move in a circular path, as shown, or in a helical or spiral path around the heart 5. The detector device comprises a detector with a large detector field, so that the entire heart 5 can be recorded by a single image. Moreover, it is also possible for partial regions of the heart 5 to be recorded, for example slices of the heart 5 with a different slice thickness. Along the circular path, three circle segments are shown which symbolize time windows 1, 2, 3 and enclose the circular path in each case by 180°, identified by the squares 1 to 3. These are referred to as Pi time windows 1, 2, 3. The circle segments are shifted with respect to one another, so that they have different start and end points along the circular path. This means that the time windows 1, 2, 3 are not rigid but rather are displaceable, and different movement states of the heart 5 are recorded. The time windows 1, 2, 3 are preferably shifted periodically and by the same distances in one direction. This is important since the heartbeat of a patient, which takes place in various heart phases, is not uniform but rather is subject to changes; the number of heartbeats per unit time and the temporal spacing between the heartbeats are subject to changes. The heart rate changes during the recording for example by introducing contrast agent into the patient's body, by causing the patient to become excited and by other effects, and the movement state of the heart 5 does not have a periodic profile. The squares in FIG. 1 which surround the time windows 1, 2, 3 represent volumes of the heart 5, and the movement of the heart 5 is deduced from the volume, represented symbolically by the size of the squares. The movement of the heart 5 is more pronounced in certain volumes of the heart 5 than in other volumes; this is explained in more detail below with reference to FIG. 2. The volumes of the heart 5 are therefore a measure of the intensity of movement of the heart 5.

On account of the change in heart rate, it has not previously been possible in the prior art to successfully modulate the tube current beforehand or prospectively in order to record only certain movement states with a given modulated tube current. In the prior art approach, undesirably different movement states are recorded and image artifacts arise. In order to reconstruct an image, at least recorded data from half a revolution of the radiation source 15 about the heart 5 are required, and specifically data which are recorded during the most restful phase of the heart 5, when the heart 5 exhibits little movement. The computer tomograph carries out a prospective modulation of the tube current of the radiation source 15; in other words, the tube current is changed in a predictive manner during the recording operation without knowing the actual following movement state of the heart 5. During the recording operation, low-resolution images are continually reconstructed from the recorded data. In this way, the movement state of the heart 5 is ascertained in the computer tomograph with the aid of the low-resolution images, preferably by comparing successive images in the computer tomograph in which similar movement states can be assigned to one another. Preferably, the low-resolution images cover only part of the heart 5, for example a slice, so that it is not the entire heart 5 which is recorded. This is referred to here as a partial image of the heart 5. The low-resolution partial images are sufficient for ascertaining different movement states of the heart 5. The low image resolution is for example around 64 voxels in both detector dimensions, and a high image resolution is for example around 512 voxels in both detector dimensions. For each image, 180° of recorded data are required from a 180° revolution of the radiation source 15 around the heart 5. A high-resolution reconstruction of the image, which leads to a desired image quality and is the aim of image reconstruction for medical applications, is carried out retrospectively after the end of the recording method, unlike the aforementioned low-resolution reconstruction. The high-resolution reconstruction of the image is carried out using a high number of voxels and the choice of a suitable filter. For high-resolution reconstruction, use is made of similar movement states with as little intrinsic movement of the heart 5 as possible, said movement states being determined in the described manner from the low-resolution images.

The tube current and consequently the radiation intensity affects the signal-to-noise (S/N) ratio and is set to be high in phases with a restful heart; a high tube current leads to a high signal-to-noise ratio. The low tube current of the X-ray tube is set in the range of around 50 mA, and the high tube current of the X-ray tube is set in the range of around 250 mA to 300 mA. Other tube currents are also possible. Overall, the transmitted radiation dose of the computer tomograph is drastically reduced by means of the change in radiation intensities. The radiation exposure for the patient and the operating staff is reduced as a result, whereas the image quality is maintained compared to methods with higher radiation exposure. A computer program is provided which is implemented in the computer tomograph, which computer program is designed to control the radiation source 15 and controls the time windows 1, 2, 3 and the tube currents of the radiation source 15, as described. The roman numerals I, II, III denote regions which are located between the time windows 1, 2, 3; the regions I, II, III and the time windows 1, 2, 3 therefore depend on one another.

As an alternative, with the overall radiation dose being maintained compared to image recording of the prior art, a further increased radiation intensity may be used within the time windows 1, 2, 3 which is higher than the described radiation intensity of high image resolution, wherein the image quality is increased. Consequently, in this variant, a higher image quality is achieved for approximately the same overall radiation dose during image recording.

FIG. 2 shows by way of example a diagram of a heartbeat in which the heart phase is plotted on the abscissa and the movement of the heart 5 is plotted on the ordinate; a variable heart volume is shown as a function of time. It can be seen that the curve firstly rises steeply, passes through a maximum, falls, passes through a minimum and then passes through two further maxima. The time windows 1, 2, 3 as shown in FIG. 1 are plotted on the curve, wherein the roman numerals I to III identify approximately the start of the respective time window 1 to 3 and are located between the time windows 1, 2, 3. As can be seen, the start of the time windows 1, 2, 3 is shifted from the first time window 1 to the third time window 3 along the curve and starts in each case at a later point in time. Preferably, images of the heart 5 are recorded with as little movement as possible in a movement phase with little variation; this requirement is best satisfied in the region of the minima and maxima of the curve. In the first time window 1, the recording of the heart 5 with a high image resolution starts at an early point in time which is far away from the first minimum of the curve shown in FIG. 2; the time at which the first time window 1 starts is therefore not selected in an optimal manner. The second time window 2 starts at a later point in time which lies closer to the minimum, which is preferably carried out at the time of little heart movement. The start of the third time window 3 is in turn located closer to the minimum of the curve shown, wherein the time window 3 includes the minimum. The shift in the time windows 1, 2, 3 is consequently carried out such that the time windows 1, 2, 3 include states of the heart phase in which the movement of the heart 5 is slight. Preferably, the heart 5 is firstly recorded with a low power of the X-ray tube and a low radiation intensity, from which an X-ray image with low resolution is produced by means of a rapid image reconstruction. This serves essentially to ascertain the specific movement state of the heart 5. For this purpose, the image reconstructed rapidly during the recording is compared with images contained in a memory device of the computer tomograph, the movement state of which is known and which exhibit little heart movement. Another possibility provides that reconstructed images of two successive time windows 1, 2, 3 are compared. If the compared images differ greatly from one another, the heart 5 is in a state of considerable movement; if, on the other hand, the compared images are similar, a state of considerable rest of the heart movement exists. From the comparison, it is possible to determine in which movement state the heart 5 to be examined is situated during the respective recordings using the time windows 1, 2, 3. The comparison data from the memory device may in this case originate from a previous time window 1, 2, 3. This measure can be used to predict the point at which the time windows 1, 2, 3 are situated with respect to the heartbeat and whether a shift in the time windows 1, 2, 3 to other movement states with little heart movement is necessary. If the ascertained movement state of the heart 5 is in a desired heart phase with little heart movement, as ascertained by the comparison, the power of the X-ray tube is then increased and an image with a higher image resolution is produced with a higher radiation intensity. If the ascertained movement state of the heart 5 is in an undesirable heart phase with considerable heart movement, during a significant rise in the curve shown in FIG. 2, the tube current remains low regardless of the position of the time windows 1, 2, 3. The tube current is increased at the correct moment while the heart 5 is more or less at rest, regardless of the point at which the time windows 1, 2, 3 are situated. The heart 5 is then irradiated with a full radiation dose with a revolution of 180° around the heart 5. The time window 1, 2, 3 may be shifted temporally forward or backward with regard to a sequence of the time windows 1, 2, 3 with equal temporal spacings. Only when a movement state of little heart movement is recorded by a time window 1, 2, 3 is the power of the X-ray tube increased and an image of higher image resolution produced with a higher radiation intensity, said image of higher image resolution being particularly suitable for further processing in order to obtain images for diagnostic purposes. In this way, in computer tomography, changes in heart rate are taken into account and images are always recorded when there is little movement of the heart 5, with image artifacts which are not very pronounced. When such a curve exists, use is preferably made of data recorded with a high radiation intensity of a radiation source 15 in similar movement states in order to reconstruct images with a high image resolution in the region of the two phase points plotted on the abscissa. In the region of the two phase points, there is little change in the volume of the heart 5; curve sections with a low rise and saddle points of the curve are located at these points. There is little movement of the heart 5 in the region where there is little change in volume, and this is therefore particularly suitable for recording image data for reconstruction purposes.

FIG. 3 shows a spiral or helical curve along which the radiation source 15 of the computer tomograph moves relative to the recorded object, the heart 5. The helical or spiral curve or path of the radiation source 15 is given by way of example; it is also possible to use a circular path of the radiation source 15 around the heart 5. This leads to different image reconstruction methods for obtaining the image in computer tomographs, as is known. In this example, the patient moves along the axis of rotation of the spiral path 13 on a patient table, and the radiation source 15 moves in a circular path about the latter, so that the illustrated spiral path 13 is obtained as the position of the radiation source 15 with respect to the heart 5. If the patient table does not move, there is a circular path of the radiation source 15. Shown in bold in FIG. 3 are the described regions I, II, III, first spiral path sections 11, between the time windows 1, 2, 3, whereas the other recording times, the time windows 1, 2, 3, are shown in dashed line, second spiral path sections 12. The first spiral path sections 11 and the second spiral path sections 12 vary according to the shift in the time windows 1, 2, 3, so that different start and end points of the regions I, II, III and of the time windows 1, 2, 3 are set along the spiral path 13.

FIG. 4 shows a further diagram of a heart movement as a function of the heart phase. At the minima of the curve, which shows the heart movement in images of the heart 5, vertical lines 14 are plotted, in the region of which for example preferred time windows 1, 2, 3 are located, since the heart movement is minimal at the points of intersection of the lines 14 with the curve and at these points an image reconstruction can be carried out with few image artifacts because only negligible image artifacts appear at the points of intersection following the image reconstruction. In one special embodiment, unlike in the above description, an electrocardiogram 10 is recorded, as shown in FIG. 5. This shows the heartbeat as a function of time and is assigned to the heart phase shown in FIG. 4. Using the electrocardiogram 10, it is possible to ascertain the time periods of the moving heart 5 at which the heart 5 is as much at rest as possible and exhibits little intrinsic movement, shown by the vertical lines 14 in the electrocardiogram 10 of FIG. 5, at which the heart 5 is almost not moving and an almost horizontal curve profile exists. Based on the electrocardiogram 10, which is recorded simultaneously by the described computer tomograph, the time windows 1, 2, 3 are set at which recordings take place with high radiation intensity. When the curve profile of the electrocardiogram 10 is more or less horizontal, in the region of the vertical lines 14, and consequently a state of little movement of the heart 5 exists, the radiation intensity of the X-ray tube is increased. When the curve profile of the electrocardiogram 10 changes, in the event of rises in the curve or peaks, the radiation intensity of the X-ray tube is reduced.

1. A method of recording images of the heart in computer tomography, in which, in order to prevent movement artifacts, the images are reconstructed on the basis of similar movement states of the heart and different radiation intensities are used for different movement states.

2. A method as claimed in claim 1, in which image data are recorded with a high radiation intensity of a radiation source in similar movement states in order to reconstruct images with a high image resolution and in the other movement states the heart is acted upon by a low radiation intensity with a correspondingly low image resolution.

3. A method as claimed in claim 1, in which similar movement states of the heart are determined on the basis of an image reconstruction of a partial image of the heart.

4. A method as claimed in claim 1, in which a low image resolution has a voxel number of around 64 voxels in both detector dimensions and a high image resolution has a voxel number of around 512 voxels in both detector dimensions.

5. A method as claimed in claim 1, in which a high radiation intensity of the radiation source is triggered to record the similar movement states for reconstructing the images by comparing the image data of various successive movement states at a low radiation intensity.

6. A method as claimed in claim 1, in which the radiation intensity is changed by changing the power of the radiation source of the computer tomograph.

7. A method as claimed in claim 6, in which the tube current is around 50 mA for a low radiation intensity of the radiation source and around 250 mA to 300 mA for a high radiation intensity.

8. A method as claimed in claim 1, in which an electrocardiogram is recorded in order to detect the different movement states of the heart and to trigger the different radiation intensities on the basis of the different movement states of the heart.

9. A method as claimed in claim 1, in which image data of the heart are recorded from a circular detector path.

10. A method as claimed in claim 1, in which image data of the heart are recorded from a helical detector path.

11. A method as claimed in claim 1, in which a prospective gating method is used.

12. A method as claimed in claim 1, in which the reconstruction of the image is carried out during the recording of the image.

13. A computer tomograph for recording images of the heart in computer tomography by means of time windows which exhibit similar movement states of the heart in order to prevent movement artifacts, said computer tomograph comprising a control device which controls a radiation source with different radiation intensities for different movement states.

14. A computer program for a computer tomograph for recording images of the heart in computer tomography by means of time windows which exhibit similar movement states of the heart in order to prevent movement artifacts, and for controlling a radiation source with different radiation intensities for different movement states.
